(19) United States
(12) Patent Application Publication
(10) Pub. No.: US 2014/0277505 A1
(43) Pub. Date: Sep. 18, 2014

(54) SPINAL IMPLANTS WITH BIOACTIVE GLASS MARKERS

(71) Applicant: Dale Mitchell, Jupiter, FL (US)

(72) Inventor: Dale Mitchell, Jupiter, FL (US)

(21) Appl. No.: 14/213,743

(22) Filed: Mar. 14, 2014

Related U.S. Application Data
(60) Provisional application No. 61/800,705, filed on Mar. 15, 2013.

(51) Int. Cl. A61L 27/10; A61B 17/70; A61L 27/06; A61F 2/44

(52) U.S. Cl. CPC A61L 27/10; A61F 2/447; A61B 17/7059; A61L 27/06
USPC 623/17.16; 606/246

(57) ABSTRACT

The present invention relates to orthopedic implants. More specifically, the present invention is a series of orthopedic implants constructed from biocompatible material, each including a plurality of markers constructed from bioactive glass material, some of which are radio-opaque. In addition to providing recognizable markers for use by the surgeon implanting the device, the bioactive glass markers provide a lattice structure which allows for the in-growth of bone into portions of the implant. The in-growth provides enhanced structural integrity between the implant and the bone structure of the patient and may shorten healing time.

### RELATED APPLICATIONS

In accordance with 37 C.F.R 1.76, a claim of priority is included in an Application Data Sheet filed concurrently herewith. Accordingly, the present invention claims priority to U.S. Provisional Patent Application No. 61/800,705, entitled “SPINAL IMPLANTS WITH BIO-ACTIVE GLASS MARKERS”, filed Mar. 15, 2013. The contents of the above referenced application are herein incorporated by reference in its entirety.

### FIELD OF THE INVENTION

The present invention and method of use relate to bone fixation devices. More particularly, the present invention relates to spinal or other medical implants having bioactive glass markers or coatings which aid in positioning of the implant as well as bone fusion.

### BACKGROUND OF THE INVENTION

A normal human spine is segmented with seven cervical, twelve thoracic and five lumbar segments. The lumbar portion of the spine resides on the sacrum, which is attached to the pelvis. The pelvis is supported by the hips and leg bones. The bony vertebral bodies of the spine are separated by intervertebral discs, which reside sandwiched between the vertebral bodies and operate as joints, allowing known degrees of flexion, extension, lateral bending and axial rotation.

The intervertebral disc primarily serves as a mechanical cushion between adjacent vertebral bodies, and permits controlled motions within vertebral segments of the axial skeleton. The disc is a multi-element system, having three basic components: the nucleus pulposus (“nucleus”), the anulus fibrosus (“anulus”) and two vertebral end plates. The end plates are made of thin cartilage overlying a thin layer of hard, cortical bone that attaches to the spongy, richly vascular, cancellous bone of the vertebral body. The plates thereby operate to attach adjacent vertebrae to the disc. In other words, a transitional zone is created by the end plates between the malleable disc and the bony vertebrae. The anulus of the disc forms the disc perimeter, and is a tough, outer fibrous ring that binds adjacent vertebrae together. The fiber layers of the anulus include fifteen to twenty overlapping plies, which are inserted into the superior and inferior vertebral bodies at roughly a 40-degree angle in both directions. This causes bi-directional torsional resistance, as about half of the angulated fibers will tighten when the vertebrae rotate in either direction. It is common practice to remove a spinal disc in cases of spinal disc deterioration, disease or spinal injury. The discs sometimes become diseased or damaged such that the intervertebral separation is reduced. Such events cause the height of the disc nucleus to decrease, which in turn causes the anulus to buckle in areas where the laminated plies are loosely bonded. As the overlapping laminated plies of the anulus begin to buckle and separate, either circumferential or radial anular tears may occur. Such disruption to the natural intervertebral separation produces pain, which can be alleviated by removal of the disc and maintenance of the natural separation distance. In cases of chronic back pain resulting from a degenerated or herniated disc, removal of the disc becomes medically necessary.

In some cases, the damaged disc may be replaced with a disc prosthesis intended to duplicate the function of the natural spinal disc. In other cases it is desired to fuse the adjacent vertebrae together after removal of the disc, sometimes referred to as “intervertebral fusion” or “interbody fusion.” In this process, spondylodesis or spondylosyndesis is used to join two or more vertebrae to eliminate pain caused by abnormal motion, degradation, fractures or deformities of the vertebrae.

Spinal plates have become one common approach to attaching one adjacent vertebra to another. A spinal plate generally includes an elongated plate of a metal such as titanium or stainless steel. The plate includes a plurality of apertures positioned to allow a surgeon to attach the plate across at least two vertebras with screws. The combination of the plate and screws serve to hold the adjacent vertebra together while the intervertebral fusion occurs.

Biomaterials have been used as implants in the field of spine, orthopedics and dentistry including trauma, fracture repair, reconstructive surgery and alveolar ridge reconstruction, for over a century. Although metal implants, such as titanium, have been the predominant implants of choice for these types of load-bearing applications, additional ceramics and non-resorbable polymeric materials have been employed within the last twenty-five years due to their biocompatibility and physical properties.

Polyetheretherketone (PEEK) is a biomaterial often used in medical implants. For example, PEEK can be molded into preselected shapes that possess desirable load-bearing properties. PEEK is a thermoplastic with excellent mechanical properties, including a Young's modulus of about 3.6 GPa and a tensile strength of about 100 MPa. PEEK is semi-crystalline, melts at about 340.degree. C., and is resistant to thermal degradation. Such thermoplastic materials, however, are not, osteoproductive, or osteoconductive.

Therefore, there is a need for a series of orthopedic implants which combine a biocompatible material or polymer such as, but not limited to, titanium or PEEK with a glass. The combination should provide the surgeon with radio opaque markers for use in positioning the implant. The radio opaque markers should be constructed of glass of various particle sizes, and have the appropriate structural and mechanical properties to withstand the stresses necessary for use in spinal and orthopedic implants. In addition, the bioactive glass should provide a lattice for bone in-growth into a portion of the implant to integrate the implant into the bone of the patient.

### SUMMARY OF THE INVENTION

The present invention relates to orthopedic implants. More specifically, the present invention is a series of orthopedic implants constructed from biocompatible material, each including a plurality of markers constructed from bio-active glass material, some of which are radio-opaque. In addition to providing recognizable markers for use by the surgeon implanting the device, the glass markers provide a lattice structure which allows for the in-growth of bone into portions of the implant. The in-growth provides enhanced structural integrity between the implant and the bone structure of the patient and may shorten healing time. In an alternative embodiment, glass is coated or impregnated into the outer surface of the implant to provide a lattice structure which allows for the in-growth of bone into portions of the implant.

Accordingly, it is an objective of the present invention to provide a series of orthopedic implants constructed of a biocompatible material having bioactive glass markers which aid in the implants insertion.

It is another objective of the present invention to provide a series of orthopedic implants constructed of a biocompatible material having bioactive glass markers wherein the markers aid in providing bone in-growth into and around the implant.

It is yet another objective of the present invention to provide a radio opaque marker constructed from bioactive glass for orthopedic implants.

It is still another objective of the present invention to provide a plurality of methods of securing a bioactive glass marker to an orthopedic implant.

It is still yet a further objective of the present invention to provide an interbody spinal implant having bioactive glass markers.

Yet another objective of the present invention is to provide a spinal plate having bioactive glass markers.

Still yet another objective of the present invention is to provide an orthopedic implant having an outer surface coated or impregnated with glass particles, some of which may be radio opaque.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a top view of a pivotable interbody spacer, according to one exemplary embodiment;

FIG. 2 is a side view of the embodiment illustrated in FIG. 1;

FIG. 3 is an end view of the embodiment illustrated in FIG. 1;

FIG. 4 is a section view taken along lines 4-4 of FIG. 1;

FIG. 5 is a section view taken along lines 5-5 of FIG. 4;

FIG. 6 is a section view taken along lines 6-6 of FIG. 2;

FIG. 7 is a top view of an alternative embodiment of the interbody spacer having an angled profile for correction of spinal deformities;

FIG. 8 is a side view of the embodiment illustrated in FIG. 7;

FIG. 9 is an end view of the embodiment illustrated in FIG. 7;

FIG. 10 is a section view taken along lines 10-10 of FIG. 7;

FIG. 11 is a section view taken along lines 11-11 of FIG. 10;

FIG. 12 is a section view taken along lines 12-12 of FIG. 8;

FIG. 13 is a perspective view of a spinal section, illustrated with an interbody spacer in the disc space;

FIG. 14 is a section view taken along lines 14-14 of FIG. 13;

FIG. 15 is a perspective view of a spinal plate according to one embodiment of the present invention;

FIG. 16 is a partial section view taken along lines 16-16 of FIG. 15 illustrating in-growth pockets containing bio-active glass markers;

FIG. 17 is a partial section view taken along lines 17-17 of FIG. 15 illustrating in-growth pockets containing bio-active glass markers;

FIG. 18 is a side view of the embodiment illustrated in FIG. 15;

FIG. 19A is a side view illustrating a pedicle screw according to one embodiment of the present invention;

FIG. 19B is an exploded view of the embodiment illustrated in FIG. 19A;

FIG. 20 is a side view of the embodiment illustrated in FIG. 19A;

FIG. 21 is a side view of the embodiment illustrated in FIG. 19A;

FIG. 22 is a perspective view of the embodiment illustrated in FIG. 19A;

FIG. 23 is a perspective view illustrating an intervertebral implant according to one embodiment of the present invention;

FIG. 24 is a top view of the intervertebral implant illustrated in FIG. 23;

FIG. 25 is a side view of the intervertebral implant illustrated in FIG. 23;

FIG. 26 is a perspective view illustrating an intervertebral implant according to one embodiment of the present invention;

FIG. 27 is a perspective view of the intervertebral implant illustrated in FIG. 26;

FIG. 28 is a side view of the intervertebral implant illustrated in FIG. 26;

FIG. 29 is a top view of the intervertebral implant illustrated in FIG. 26;

FIG. 30 is a side view of the intervertebral implant illustrated in FIG. 26;

FIG. 31 is a perspective view illustrating an intervertebral implant according to one embodiment of the present invention;

FIG. 32 is a top view of the intervertebral implant illustrated in FIG. 31;

FIG. 33 is a side view of the intervertebral implant illustrated in FIG. 31;

FIG. 34 is a perspective view illustrating an intervertebral implant according to one embodiment of the present invention;

FIG. 35 is a top view of the intervertebral implant illustrated in FIG. 34.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Referring to FIGS. 1-6, which are now referenced, one embodiment of the interbody spacer 100 is illustrated. As illustrated, the present exemplary interbody spacer is designed for use as an intervertebral spacer in spinal fusion surgery, where portions of an affected disc are removed from between two adjacent vertebrae 102 and replaced with an interbody spacer 100 that provides segmental stability, may correct a deformity, and allows for bone to grow between the two vertebrae to bridge the gap created by disk removal (FIG. 13).

As shown, the present exemplary interbody spacer 100 has a generally rectangular shape comprised of a pair of side rails 104, a pair of cross supports 106, 107 and a transverse spindle 108 to facilitate the insertion of the interbody spacer through a narrow approach window into the disk space. As illustrated, the side rails 104 and cross supports 106, 107 are constructed to include bio-active glass markers 119 held within pockets 118. The markers and pockets are arranged to provide a visual indicator to a surgeon inserting the device, indicating the orientation of the interbody spacer 100. In the preferred embodiment, the markers are cylindrical in shape to fit within the pockets 118. The markers may be sized for a press fit, or alternatively a biocompatible adhesive may be utilized to retain the markers within the pockets. In alternative embodiments, locking tapers or mechanical mechanisms including biocompatible shrink wrap (not shown) may be utilized to retain the markers in place for insertion. While the basic preferred embodiment of the interbody spacer 100 is preferably constructed from biocompatible material such as polyetheretherketone (PEEK), polyaryletherketone (PEAK), stainless steel, titanium or the like, the markers are preferably constructed from a bioactive glass having a composition such as that found in 45S5 and 13-93 glasses made by Mo-Sci Corporation of Rolla, Mo. It should be noted that in some embodiments these compositions are constructed and arranged to be radio opaque bioactive glass. It should also be noted that other bioactive glass materials may be utilized without departing from the scope of the invention; such bioactive glass compositions may include, but should not be limited to, 55SF, S53P4, Trubone and Osteofelt also produced by Mo-Sci Corporation of Rolla, Mo. These glasses may be produced to include micro-spheres, powders, chopped or continuous glass fibers. The glass may include enhanced bone growth properties or antibacterial properties which includes antimicrobial and single cell organisms. It should also be noted that while the markers of the preferred embodiment include a length and diameter that would position a top surface of the marker below the top surface of the pocket 118 as illustrated in FIGS. 1-6, the marker may include a length that would cause the marker to extend beyond the distal edges of the implant as illustrated in FIGS. 7-12 without departing from the scope of the invention.

Still referring to FIGS. 1-6, the interbody spacer 100 includes a proximal end 112 that will be closest to a surgeon during use, and a distal end 114 that will likely be the leading edge of insertion during use. In general, the proximal end 112 is constructed and arranged for connection to an insertion tool that allows the interbody spacer to be grasped or locked into a specific orientation with respect to the insertion tool. In a most preferred embodiment, the insertion tool is constructed and arranged to include a grasping mode which allows rotation of the implant about a spindle axis, and a locking mode that allows the implant to be locked into the desired orientation once the implant is positioned in the desired orientation. This engagement is sufficiently rigid to allow the surgeon to strike the insertion tool when necessary without disturbing the orientation yet allows the surgeon to reposition the interbody spacer as many times as desired without completely releasing the implant by utilizing the grasping mode. In the illustrated embodiment, the distal end 114 of the interbody spacer 100 has a double elliptical leading edge for ease of insertion through the overlying tissues and into the intervertebral space.

The central portion of the interbody spacer 100 may have a variety of apertures, bores and/or cavities 110 designed to facilitate and support bone growth. The apertures are particularly useful for containing bone growth enhancement materials such as, but not limited to, glass, bone chips or fragments, bone morphogenic protein (BMP), bone cement or the like. In this manner, the bone growth enhancement materials may be delivered directly to the disc space. According to one embodiment, the side rails and cross supports of the interbody spacer are hollowed out to increase cavity volume while maintaining surface area in contact with the bone to prevent the interbody spacer from impacting into the bone. Consequently, the present exemplary interbody spacer 100 employs geometry that provides for a compact interbody spacer with relatively large surface area and internal cavity 110. Other cavities and geometries may be included in the interbody spacer structure, such as a hollow transverse spindle 108.

According to one exemplary embodiment, the interbody spacer 100 has an upper face 124 and an opposing lower face 126. A series of ridges 128 traverse the upper and lower faces 124, 126. Pockets 118 are dispersed throughout the ridges and troughs for containing the bioactive glass material. The ridges 128 are configured to facilitate the insertion of the interbody spacer 100 by preventing retrograde motion and slippage during the insertion process. After the surgery is complete, the bioactive glass markers 119 positioned between the ridges 128 also may provide increased surface area, encourage bone growth, and/or prevent dislocation of the interbody spacer 100. In a most preferred embodiment, each ridge 128 includes a substantially vertical face 129 and an angled face 130 wherein the pockets 118 are positioned along the angled face. This construction allows the interbody spacer to be easily pushed or tamped into position while resisting rearward migration. In a preferred embodiment, two markers are positioned relative to the transverse spindle 108, three markers relative to the center cross support 106 and two relative to the leading cross support 107.

Referring to FIGS. 7-12, an alternative embodiment of the interbody spacer 300 is illustrated. This embodiment is similar to the embodiment illustrated in FIGS. 1-6 with the exception that the upper and lower faces 124, 126 are arranged to include a face angle 116 with respect to each other so that one side rail 104 is taller than the other. This construction allows the surgeon to correct spinal deformities such as lordosis, scoliosis or the like. It should also be noted that this embodiment illustrates the bioactive glass markers 119 extending beyond the outer surface of the pocket 118.

Referring to FIGS. 13 and 14, the interbody spacer 100 is illustrated in position between a pair of vertebrae 102. While the present interbody spacer may be utilized anywhere along the spine, the axis of rotation along the centerline of the transverse spindle 108 makes the device particularly suited for use in the lower spine, most particularly between the L-2 and S-1 disc spaces. FIG. 14 is a partial perspective view of FIG. 13 illustrated with the upper vertebrae removed for clarity, further illustrating the positioning and the cooperation between the upper and lower faces 124, 126 with the bone.

The present exemplary device and unique method provide for a pivotable interbody spacer that provides a user with the ability to insert the interbody spacer in a non-linear path. The insertion instrument can lock onto the interbody spacer at multiple angles to allow for the interbody spacer to be pivoted in increments if the instrument rotation is restricted such that the instrument can only be rotated less than the total rotation required to position the interbody spacer. This additional surgical flexibility can allow insertion of the interbody spacer with the removal of less tissue and bone which results in less invasive surgery, fewer post operative complications, and quicker patient recovery time.

Referring to FIGS. 15-18, a spinal plate assembly 30 including bio-active glass markers 119 is illustrated. The spine plate assembly 30 generally includes a spine plate 32, a locking member 34, a plurality of bone screws 36 and a plurality of markers 119. The spine plate 32 is preferably constructed from a biocompatible material such as titanium, and includes a bottom surface 38, a top surface 40, a pair of side surfaces 42 and a pair of end surfaces 44. At least two bores 46 extend through the top and bottom surfaces 38, 40, each of the bores are sized for passage of a bone screw 36. In addition, each bore 46 includes a counterbore 48 extending downwardly from the top surface 40. The counterbore is sized and shaped to substantially contain a head portion 50 of the bone screw. The counterbore may be of any shape desirable to match with the bone screw. For example, the counter bore may be spherical, square, truncated or any suitable combination thereof. A segmented T-slot 52 extends between the pair of end surfaces 44 and substantially parallel to the top surface 40. A first leg 54 of the T-slot extends through the top surface 40 while portions of the second and third legs 56, 58 extend into each counterbore 48. The segments of the T-slot 52 are separated by sight windows 60 extending between the top and bottom surfaces. The sight windows 60 aid the surgeon in placement of the spinal plate 32 by allowing the surgeon to view anatomical features through the plate. The spinal plate also preferably includes at least one, and more preferably two anchor pockets 62. The anchor pockets are generally constructed and arranged to cooperate with a portion of the locking member 34 to secure the locking member to the spinal plate. The anchor pockets 62 extend downward from the top surface 40 to about the same depth as the second and third legs 56, 58 of the T-slot 52 and are wider than the T-slot 52 when viewed from an end surface 44 of the spinal plate 32. The anchor pockets 62 include side surfaces 64 and end surfaces 66 which cooperate with the locking member 34. The spinal plate 32 may additionally include tool apertures 68 which aid in the placement of the plate. The tool apertures 68 are preferably sized for cooperation with a gripping tool or K-wire, whereby the plate may be more easily maneuvered into position within the anatomy of a human or animal in vivo. The tool aperture may additionally function as windows for the surgeon once the plate has been maneuvered into position.

As illustrated, the bottom surface 38 is constructed to include bioactive glass markers 119 held within pockets 118. The markers and pockets are arranged to provide a visual indicator to a surgeon inserting the device, indicating the orientation of the interbody body spacer 100. In the preferred embodiment, the markers are cylindrical in shape to fit within the pockets 118. The markers may be sized for a press fit, or alternatively a biocompatible adhesive may be utilized to retain the markers within the pockets. In alternative embodiments, locking tapers or mechanical mechanisms including biocompatible shrink wrap (not shown) may be utilized to retain the markers in place for insertion. While the basic preferred embodiment of the plate assembly 30 is preferably constructed from biocompatible material such as titanium, stainless steel, shape memory alloy or the like, the markers are preferably constructed from a bioactive glass having a composition of 45S5 and 13-93 glasses made by Mo-Sci Corporation of Rolla, Mo. It should be noted that some embodiments of these compositions are constructed and arranged to be radio opaque bioactive glass. It should also be noted that other bioactive glass materials may be utilized without departing from the scope of the invention; such bioactive glass compositions may include, but should not be limited to 55SF, S53P4, Trubone and Osteofelt also produced by Mo-Sci Corporation of Rolla, Mo. These glasses may be produced to include micro-spheres, powders, chopped or continuous glass fibers. It should also be noted that while the markers of the preferred embodiment include a length and diameter that would position a top surface of the marker below the top surface of the pocket 118 as illustrated in FIGS. 1-6, the marker may include a length that would cause the marker to extend beyond the distal edges of the implant as illustrated in FIGS. 16-18 without departing from the scope of the invention. It should also be noted that the top surface of the bioactive marker may include a rounded, pointed, truncated or other suitable shape that is constructed and arranged to cooperate with the underlying bone of the patient. In this manner, the markers may serve to hold the implant in position prior to the insertion of fasteners.

Referring to FIGS. 19A-22, an alternative embodiment employing the teachings of the present invention is illustrated herein as a polyaxial pedicle screw 200. The pedicle screw 200 includes a shaft portion 202 having a spherical head portion 204 which cooperates with a tulip portion 206 to allow polyaxial movement therebetween, as is known in the art. In this embodiment, the shaft portion 202 includes a plurality of cross drilled apertures or pockets 208 sized to accept bioactive glass markers 119. The markers and pockets are arranged to provide a visual indicator to a surgeon inserting the device, indicating the orientation of the interbody body spacer 100. In the preferred embodiment, the markers are cylindrical in shape to fit within the pockets 118. The markers 119 may be sized for a press fit, or alternatively a biocompatible adhesive may utilized to retain the markers within the pockets. In alternative embodiments, locking tapers or mechanical mechanisms including biocompatible shrink wrap (not shown) may be utilized to retain the markers in place for insertion. While the basic preferred embodiment of the pedicle screw 200 is preferably constructed from biocompatible material such as stainless steel, titanium or the like, the markers are preferably constructed from a bioactive glass having a composition such as that found in 45S5 and 13-93 glasses made by Mo-Sci Corporation of Rolla, Mo. It should be noted that some embodiments of these compositions are constructed and arranged to be radio opaque bioactive glass. It should also be noted that other bioactive glass materials may be utilized without departing from the scope of the invention; such bioactive glass compositions may include, but should not be limited to 55SF, S53P4, Trubone and Osteofelt also produced by Mo-Sci Corporation of Rolla, Mo. These glasses may be produced to include micro-spheres, powders, chopped or continuous glass fibers. It should also be noted that while the markers of the preferred embodiment include a length and diameter that would position a top surface of the marker below the top surface of the pocket 208 as illustrated in FIGS. 19A-20, the marker may include a length that would cause the marker to extend beyond the distal edges of the shaft as illustrated in FIG. 21 without departing from the scope of the invention. It should also be noted that while the markers are the preferred embodiment, portions of the outer surface of the shaft or tulip portions may be coated or impregnated with glass particles or fibers without departing from the scope of the invention. The glass may be adhered or otherwise impregnated into the outer surface by any means known in the art for coating materials.

Referring to FIGS. 23-25, an alternative embodiment of an intervertebral spacer 300 is illustrated. The intervertebral spacer has a generally rectangular shape comprised of a pair of side rails 304, a pair of cross supports 306 and a threaded bore 308 to facilitate the insertion of the intervertebral spacer through a narrow approach window into the disk space. As illustrated, the side rails 304 and cross supports 306 are constructed to include bioactive glass markers 119 held within pockets 118. The markers and pockets are arranged to provide a visual indicator to a surgeon inserting the device, indicating the orientation of the intervertebral spacer 300. In the preferred embodiment, the markers are cylindrical in shape to fit within the pockets 118. The markers may be sized for a press fit, or alternatively a biocompatible adhesive may utilized to retain the markers within the pockets. In alternative embodiments, locking tapers or mechanical mechanisms including biocompatible shrink wrap (not shown) may be utilized to retain the markers in place for insertion. While the basic preferred embodiment of the intervertebral spacer 300 is preferably constructed from biocompatible material such as polyetheretherketone (PEEK), polyaryletherketone (PEAK), stainless steel, titanium or the like, the markers are preferably constructed from a bioactive glass having a composition such as that found in 45S5 and 13-93 glasses made by Mo-Sci Corporation of Rolla, Mo. It should be noted that some embodiments of these compositions are constructed and arranged to be radio opaque bioactive glass. It should also be noted that other bioactive glass materials may be utilized without departing from the scope of the invention; such bioactive glass compositions may include, but should not be limited to 55SF, S53P4, Trubone and Osteofelt also produced by Mo-Sci Corporation of Rolla, Mo. These glasses may be produced to include micro-spheres, powders, chopped or continuous glass fibers. It should also be noted that while the markers of the preferred embodiment include a length and diameter that would position a top surface of the marker below the top surface of the pocket 118 as illustrated in FIGS. 23-24, the marker may include a length that would cause the marker to extend beyond the distal edges of the implant as illustrated in FIG. 25 without departing from the scope of the invention. It should also be noted that while the glass markers are the preferred embodiment, portions of the outer surface of the intervertebral spacer may be coated or impregnated with glass particles or fibers without departing from the scope of the invention. The glass may be adhered or otherwise impregnated into the outer surface by any means known in the art for coating materials.

Referring to FIGS. 26-30, an alternative embodiment of the interbody spacer 400 is illustrated. In this embodiment the glass markers are replaced with elongated glass rods. The glass rods are preferably positioned along the longitudinal length of the intervertebral implant 400 so that the outer diameter of the elongated rod is below the top surface of the teeth 404 but above the root of the teeth 406 to expose the side portion of the elongated rod(s).

Referring to FIGS. 31-33, an alternative embodiment of the interbody spacer 500 is illustrated. The interbody spacer 500 has a generally rectangular shape comprised of a pair of side rails 504, a pair of cross supports 506 and an aperture 508 combined with a keyslot 510 and a pair of apertures 512 to facilitate the insertion of the interbody spacer into the disk space. As illustrated, the side rails 504 and cross supports 506 are constructed to include glass markers 119 held within pockets 118. The markers and pockets are arranged to provide a visual indicator to a surgeon inserting the device, indicating the orientation of the interbody body spacer 500. In the preferred embodiment, the markers are cylindrical in shape to fit within the pockets 118. The markers may be sized for a press fit, or alternatively a biocompatible adhesive may utilized to retain the markers within the pockets. In alternative embodiments, locking tapers or mechanical mechanisms including biocompatible shrink wrap (not shown) may be utilized to retain the markers in place for insertion. While the basic preferred embodiment of the interbody spacer 100 is preferably constructed from biocompatible material such as polyetheretherketone (PEEK), polyaryletherketone (PEAK), stainless steel, titanium or the like, the markers are preferably constructed from a bioactive glass having a composition such as that found in 45S5 and 13-93 glasses made by Mo-Sci Corporation of Rolla, Mo. It should be noted that some embodiments of these compositions are constructed and arranged to be radio opaque bioactive glass. It should also be noted that other bioactive glass materials may be utilized without departing from the scope of the invention; such bioactive glass compositions may include, but should not be limited to 55SF, S53P4, Trubone and Osteofelt also produced by Mo-Sci Corporation of Rolla, Mo. These glasses may be produced to include micro-spheres, powders, chopped or continuous glass fibers. It should also be noted that while the markers of the preferred embodiment include a length and diameter that would position a top surface of the marker below the top surface of the pocket 118 as illustrated in FIGS. 31-32, the marker may include a length that would cause the marker to extend beyond the distal edges of the implant as illustrated in FIG. 33 without departing from the scope of the invention. It should also be noted that while the glass markers are the preferred embodiment, portions of the outer surface of the intervertebral spacer may be coated or impregnated with glass particles or fibers without departing from the scope of the invention. The glass may be adhered or otherwise impregnated into the outer surface by any means known in the art for coating materials.

Referring to FIGS. 34-35, an alternative embodiment of the interbody spacer 600 is illustrated. In this embodiment the glass markers are replaced with elongated glass rods 602. The glass rods are preferably positioned along the longitudinal length of the intervertebral implant 600 so that the outer diameter of the elongated rod is below the top surface of the teeth 604 but above the root of the teeth 606 to expose the side portion of the elongated rod(s).

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bone stabilizing implant comprising:
an implant for implantation into an animal, said implant including a plurality of surfaces, at least one of said plurality of surfaces being a bone contacting surface whereby a portion of said bone contacting surface contacts a bone within said animal when secured in an implanted position, said bone contacting surface of said implant being at least partially constructed of bioactive glass.

2. The bone stabilizing implant of claim 1 wherein said bioactive glass is coated onto said bone contacting surface, a substrate of said bone contacting surface being constructed from a different material than said bioactive glass.

3. The bone stabilizing implant of claim 2 wherein said bioactive glass is impregnated into said bone contacting surface.

4. The bone stabilizing implant of claim 1 wherein at least a portion of said bioactive glass is formed to include micro-spheres.

5. The bone stabilizing implant of claim 1 wherein at least a portion of said bioactive glass is formed to include powder.

6. The bone stabilizing implant of claim 1 wherein at least a portion of said bioactive glass is formed to include continuous glass fibers.

7. The bone stabilizing implant of claim 1 wherein at least a portion of said bioactive glass is formed to include chopped glass fibers.

8. The bone stabilizing implant of claim 1 wherein said implant includes a biocompatible shrink wrap for securing said bioactive glass coating in position.

9. The bone stabilizing implant of claim 1 wherein said implant includes a biocompatible adhesive for securing said bioactive glass coating in position.

10. The bone stabilizing implant of claim 1 wherein said bioactive glass on said bone contacting surface is positioned within pockets, said pockets extending inwardly from said bone contacting surface toward a center portion of said implant.

11. The bone stabilizing implant of claim 10 wherein said pocket is filled with said bioactive glass to a level that is about even with said bone contacting surface.

12. The bone stabilizing implant of claim 10 wherein said pocket is filled with said bioactive glass to a level that is above said bone contacting surface.

13. The bone stabilizing implant of claim 10 wherein said pockets are constructed and arranged to provide a visual indicator to a surgeon inserting said implant, whereby said visual indicator indicates the orientation of implant.

14. The bone stabilizing implant of claim 13 wherein said bioactive glass is radio opaque.

15. The bone stabilizing implant of claim 1 wherein said bioactive glass is constructed and arranged to promote bone growth.

16. The bone stabilizing implant of claim 1 wherein said bioactive glass is constructed and arranged to be antibacterial.

17. The bone stabilizing implant of claim 1 wherein said implant is a spinal implant.

18. The bone stabilizing implant of claim 17 wherein said spinal implant is predominantly constructed from polyetheretherketone.

19. The bone stabilizing implant of claim 17 wherein said spinal implant is predominantly constructed from polyaryletherketone.

20. The bone stabilizing implant of claim 17 wherein said spinal implant is predominantly constructed from titanium.
